(19) United States
(12) Patent Application Publication
(10) Pub. No.: US 2013/0237557 A1
(43) Pub. Date: Sep. 12, 2013

(54) TREATMENT

(71) Applicants: Rajeshwar P. Mookerjee, London (GB); Vikram Sharma, London (GB); Rajiv Jalan, London (GB)

(72) Inventors: Rajeshwar P. Mookerjee, London (GB); Vikram Sharma, London (GB); Rajiv Jalan, London (GB)

(73) Assignee: UCL BUSINESS PLC, London (GB)

(21) Appl. No.: 13/816,708

(22) PCT Filed: Aug. 12, 2011
(86) PCT No.: PCT/GB11/01217

§ 371 Date: May 21, 2013

(30) Foreign Application Priority Data
Aug. 12, 2010 (GB) 1013573.9

(51) Int. Cl. A61K 31/4375; G01N 33/68; A61K 31/4178

(52) U.S. Cl. CPC A61K 31/4375; A61K 31/4178; G01N 33/6893
USPC 514/280; 514/397; 435/7.1

(57) ABSTRACT

The present invention derives from the finding that increased levels of alpha 2a adrenergic receptors (ADRA2a) are associated with chronic liver disease and that by decreasing ADRA2a levels in vivo, a number of symptoms and consequences of chronic liver disease may be reduced. Accordingly, the invention provides ADRA2a antagonists for use in a method of treating an individual suffering from liver disease.

### FIELD OF THE INVENTION

The present invention derives from the unexpected finding that expression of alpha 2a adrenergic receptor (ADRA2a) in the liver is increased in bile duct ligated rats (a model for cirrhosis). Furthermore, by antagonising ADRA2a, many of the unwanted consequences or symptoms of cirrhosis, such as portal hypertension, may be reduced. The present invention utilises these findings to identify and provide ADRA2a antagonists that may be used in the treatment of liver disease, for example in the treatment of portal hypertension.

### BACKGROUND TO THE INVENTION

Statistics from the NIH for the period 1976-80 suggest that deaths from liver cirrhosis in the US were greater than 26,000. If this data is extrapolated to incorporate the increasing burden of viral and alcoholic liver disease currently in The West and also in the under-developed world, the number exceeds millions of cases per year world-wide. This figure is likely to continue to increase with the recognition of the new entity of non-alcoholic fatty liver disease (in association with diabetes and the metabolic syndrome) which is increasingly recognized as a chronic liver disease with risk of progression.

Cirrhosis is associated with severe morbidity and mortality largely from portal hypertension. Increased blood pressure in the portal blood vessels may result from either increased volume of blood flowing through the vessels or/and increased resistance to the blood flow through the liver. In Western countries, the most common cause of portal hypertension is increased resistance to blood flow caused by extensive scarring of the liver in cirrhosis, which is most often due to chronic excessive alcohol intake.

Portal hypertension leads to the development of new veins (called collateral vessels) that directly connect the portal blood vessels to the general circulation, bypassing the liver. Because of this bypass, substances (such as toxins) that are normally removed from the blood by the liver can pass into the general circulation. Collateral vessels develop in particular at the lower end of the esophagus and at the upper part of the stomach. Here, the vessels can become variceal. These engorged variceal vessels are fragile and prone to bleeding, sometimes seriously and occasionally with fatal results.

Current treatment to lower portal pressure to decrease risk from variceal bleeding is limited to about 40% efficacy, in part due to tolerability of agents such as beta-blockers. Moreover, there is a suggestion that such agents decrease liver perfusion which may further compromise liver function, as hepatic blood flow is already low in cirrhosis, despite systemic vasodilatation.

### SUMMARY OF THE INVENTION

Increased sympathetic tone plays a pivotal role in modulating the severity of intrahepatic resistance in cirrhosis. The inventors have shown that there is a close relationship between the activation of the sympathetic nervous system, inflammatory response and the severity of portal hypertension. In particular, the present invention is based on the unexpected finding that modulation of alpha 2a adrenergic receptor (ADRA2a)-mediated sympathetic tone with an ADRA2a antagonist significantly improves systemic hemodynamics and reduces portal pressure, whilst also increasing hepatic blood flow. This observation is the opposite of what one would have expected. In cirrhosis, sympathetic activation is a consequence of splanchnic vasodilation and acts to compensate for reduced blood pressure. Therefore, the systemic circulation becomes dependent upon increased sympathetic tone. Therefore, antagonising the sympathetic nervous system would be expected to be associated with further vasodilation and a reduction in blood pressure which would limit its usefulness in cirrhosis.

Accordingly, the invention provides an antagonist of alpha 2a adrenergic receptors (ADRA2a) for use in a method of treating an individual suffering from liver disease.

The individual may be suffering from chronic liver disease, such as from liver cirrhosis. The individual may be suffering from a variety of symptoms associated with liver disease, such as peripheral vasodilation, splanchnic vasodilation, reduced cardiac output; portal hypertension; reduced mean arterial pressure; reduced hepatic arterial blood flow; increased intra-hepatic resistance; increased plasma ammonia; renal dysfunction, increased brain water; increased plasma creatinine; increased plasma lactate; increased plasma alanine and/or aspartate aminotransferases, alcoholic liver disease and/or non-alcoholic fatty liver disease.

The ADRA2a antagonist may be used to treat or prevent liver failure, such as liver failure in an individual having cirrhosis. The ADRA2a antagonist may lead to decreased expression of ADRA2a in the liver of the individual; and/or decreased levels of ADRA2a in the liver of the individual; and/or decreased ADRA2a activity such as signalling via ADRA2a in the liver of the individual. The antagonist may be (a) a specific antagonist of ADRA2a; (b) not an antagonist of ADRA2b; (c) not an antagonist of ADRA2c; (d) not an antagonist of ADRA1; and/or (e) not an antagonist of ADRB. The antagonist may be selected from BRL-44408, Yohimbine, Rauwolscine.

Thus, the present invention also relates to a method of treating liver disease in an individual in need thereof, said method comprising a step of administering to said individual an antagonist of alpha 2a adrenergic receptor (ADRA2a). Similarly, the present invention also relates to the use of an ADRA2a antagonist in the manufacture of a medicament for use in the treatment of an individual suffering from liver disease.

The invention also provides a method of identifying an agent suitable for use in treating liver disease, the method comprising determining whether a test agent is capable of decreasing the amount of ADRA2a activity such as the amount of signalling via ADRA2a, wherein the ability to decrease the amount or such ADRA2a activity indicates that the compound may be suitable for use in treating liver disease. Such a method may be carried out by assessing the amount of ADRA2a activity such as signalling via ADRA2a in cells or a tissue that expresses or contains ADRA2a. For example, the amount or activity of ADRA2a may be assessed in the liver or in tissue or cells derived from the liver, in the kidney or heart or cells derived from the kidney or heart; in inflammatory cells, platelets or neurons; or in another cell or tissue that expresses ADRA2a. Such a method may be carried out by administering a test agent to a bile duct ligated rat and determining whether the presence of the test agent leads to a decrease in the amount or activity of ADRA2a in the liver of the rat.

### BRIEF DESCRIPTION OF THE DRAWINGS

FIG. 1 reports the relationship of various factors with noradrenaline levels. FIG. 1A reports the relationship between systemic inflammatory response (SIRS) and noradrenaline levels. FIG. 1B reports the relationship between portal pressure (HVPG), hepatic blood flow and noradrenaline levels. The palest circles show measurements from compensated patients, the next set of circles show measurements from decompensated patients and the darkest circles show measurements from patients with acute on chronic liver failure (ACLF). FIG. 1C reports the relationship between intrahepatic resistance and noradrenaline levels. This demonstrates an excellent correlation.

FIG. 2 reports the effects of treatment with BRL-44408 (BRL) or placebo (saline, N/S) in bile duct ligated (BDL) or sham operated (SHAM) rats. A: mean arterial blood pressure; B: cardiac output; C: portal pressure; D: hepatic arterial blood flow; E: intra-hepatic resistance; F: plasma ammonia levels; G: brain water; H: plasma creatinine; I: plasma lactate; J: plasma aspartate aminotransferase (AST); K: plasma alanine aminotransferase (ALT); L: expression of nuclear factor κ B (NFκB) in liver tissue.

FIGS. 3 and 4 report the expression of alpha 2a receptor in the liver of sham operated rat and bile duct ligated rat. FIG. 4A=sham operated rat. FIG. 4B=bile duct ligated rat.

### DETAILED DESCRIPTION OF THE INVENTION

The sympathetic nervous system (SNS) is responsible for up- and down-regulating many homeostatic mechanisms in living organisms. Fibres from the SNS innervate tissues in almost every organ system, providing at least some regulatory function to things as diverse as pupil diameter, gut motility, and urinary output.

Noradrenaline (or norepinephrine) is a catecholamine with multiple roles including as a hormone and a neurotransmitter. Norepinephrine performs its actions on the target cell by binding to and activating adrenergic receptors. The target cell expression of different types of receptors determines the ultimate cellular effect, and thus norepinephrine has different actions on different cell types.

There are two main groups of adrenergic receptors, α and β, with several subtypes.

α receptors have the subtypes α₁ (a G_{q} coupled receptor) and α₂ (a Gᵢ coupled receptor).

β receptors have the subtypes β₁, β₂ and β₃. All three are linked to Gₛ proteins (although β₂ also couples to Gi).

Alpha-2-adrenergic receptors are members of the G protein-coupled receptor superfamily. They include 3 highly homologous subtypes: alpha 2a, alpha 2b, and alpha 2c. These receptors have a critical role in regulating neurotransmitter release from sympathetic nerves and from adrenergic neurons in the central nervous system. Studies in mouse revealed that both the alpha 2a and alpha 2c subtypes were required for normal presynaptic control of transmitter release from sympathetic nerves in the heart and from central noradrenergic neurons; the alpha 2a subtype inhibited transmitter release at high stimulation frequencies, whereas the alpha 2c subtype modulated neurotransmission at lower levels of nerve activity.

The alpha 1 adrenergic receptors are postsynaptic receptors couple with the G_{αq} G-protein to stimulate phospholipase C and the IP₃-calcium pathway that promotes vasoconstriction. The alpha 2 adrenergic receptor couples with the Gᵢ G-protein with inhibitory effects on adenyl cylase, they promote vasorelaxation and lower blood pressure.

Gᵢ alpha subunit (or Gᵢ/G₀ or Gi protein) is a heterotrimeric G protein subunit that inhibits the production of cAMP from ATP. This alpha subunit is the receptor of interest. It is located in Chromosome 7q21. G_{αq} G-protein normally couples with alpha 1 adrenergic receptor. However there are many drugs like oxymetazoline which might act as an antagonist for alpha 1 receptor and antagonist for the alpha 2 adrenergic receptor.

The present invention lies in the finding that targeting of the alpha 2a adrenergic receptor has particular utility in patients suffering from liver disease such as cirrhosis. Thus, by using an inhibitor of alpha 2a adrenergic receptor function, or an alpha 2a adrenergic receptor antagonist, symptoms associated with liver disease, such as symptoms associated with cirrhosis and portal hypertension, may be reduced or relieved.

### ADRA2a Antagonists

The present invention relates to the antagonism of alpha 2a adrenergic receptors (ADRA2a). An antagonist of ADRA2a may be any compound or molecule that inhibits or decreases the activity, function or amount of ADRA2a. Preferably the antagonist functions in cells, tissues or organs that express ADRA2a such as in the liver of the patient. The antagonist may act preferentially in the liver or may act at a number of locations including the liver. The antagonist may act preferentially in particular cell types such as inflammatory cells, platelets or neurons. Preferably the antagonist leads to a decrease in ADRA2a activity, function or amount in the cells, tissues or organs of an individual to whom the antagonist is administered, such as in one of more of the liver, kidneys, brain, and the heart of the individual. The decrease in activity may be a decrease in signalling via ADRA2a. The antagonist may be targeted to the liver or other organs, cells or tissues such as those listed above during administration as discussed further below.

Preferred antagonists are those that decrease the activity (e.g. signalling via ADRA2a) or amount (e.g. expression level measured as mRNA or protein level) of ADRA2a by at least 10%, at least 20%, at least 30%, at least 40%, at least 50%, at least 60%, at least 70%, at least 80% or at least 90% compared to the amount seen in the absence of the antagonist. For example, decreases of these sizes may be seen in the liver or liver tissue of a subject to whom the agonist has been administered. Decreases of these sizes may be seen in other tissues or organs of the individual, such as in the kidney and/or heart of the individual.

An antagonist of ADRA2a may reduce the activity or amount of ADRA2a to an amount or activity that is the same, similar to, or equivalent to, that seen in an individual not suffering from liver disease. For example, as explained herein, the expression of ADRA2a is found to be increased in association with a model of cirrhosis. Use of an ADRA2a antagonist in accordance with the present invention may lead to a reduction in ADRA2a expression in the liver of the individual being treated to a normal level, such as a level that would be seen or would be expected in an individual not suffering from chronic liver disease or cirrhosis.

The antagonist may act specifically or selectively to antagonise ADRA2a. The terms specific and selective are used interchangeably herein to refer to an effect in relation to ADRA2a in preference to an effect on other adrenergic receptors. That is, the effect of the antagonist on ADRA2a may be greater than any other biological effect of the antagonist. Such an antagonist may be specific to the inhibition of ADRA2a, that is it may decrease the activity of ADRA2a, but not other adrenergic receptors. By activity of ADRA2a herein is meant, for example, the signalling of ADRA2a. Such an antagonist may additionally or alternatively be specific to the expression of ADRA2a, that is it may decrease the expression of ADRA2a but not other adrenergic receptors.

A specific antagonist for use in accordance with the present invention may be an antagonist of ADRA2a as described herein, that does not act as an antagonist of other adrenergic receptor types such as ADRA2b, ADRA2c, ADRA1 and/or ADRB. A specific antagonist for use in accordance with the present invention may act on ADRA2a in preference to other adrenergic receptor types. For example, an antagonist of ADRA2a for use in accordance with the present invention may have one or more of the characteristics of an ADRA2a antagonist as described herein, such as this ability to reduce ADRA2a expression or signalling via ADRA2a, but may not have such characteristics in relation to other adrenergic receptor types, or may have such characteristics to a lower level in relation to other adrenergic receptor types when compared to ADRA2a. For example, an antagonist that decreases the activity (e.g. signalling via that receptor) or expression of ADRA2a may not decrease the activity (e.g. signalling via that receptor) or expression of one or more other adrenergic receptor types, or may decrease the activity of the other adrenergic receptor type(s) to a lesser extent than for ADRA2a. The lesser extent may be measured as, for example, a lower percentage decrease when compared to the activity or expression in the absence of the antagonist, such as a decrease in activity or expression of less than 30%, less than 20%, less than 10%, less than 5%, less than 2%, less than 1% or less than 0.5% when compared to the activity or expression in the absence of the antagonist or when compared to its effect on ADRA2a. An antagonist that decreases the activity, expression or amount of ADRA2a may therefore not decrease the expression or amount of other adrenergic receptor types, or may decrease the expression of other adrenergic receptor types to a lesser extent, such as a lower percentage decrease, than its effect on ADRA2a.

By other adrenergic receptor types herein is meant any adrenergic receptor that is not an alpha 2a adrenergic receptor. For example, the other adrenergic receptor type may be one or more of an alpha 2b adrenergic receptor (ADRA2b), an alpha 2c adrenergic receptor (ADRA2c), an alpha 1 adrenergic receptor (ADRA1), or a beta adrenergic receptor (ADRB) such as a beta1, beta2 or beta3 adrenergic receptor.

The other adrenergic receptor may be any one of these adrenergic receptor types. The ADRA2a antagonist may be specific to ADRA2a as discussed above in comparison to its effects on any other adrenergic receptor type. For example, the ADRA2a antagonist may be specific to ADRA2a in comparison to ADRA1.

The other adrenergic receptor may be more than one of these adrenergic receptor types. For example, the effects of the antagonist on an ADRA2a receptor may be specific, as discussed above, when compared to the effects of that agent on beta adrenergic receptors, when compared to the effects of that agent on alpha1 adrenergic receptors, and/or when compared to the effects of that agent when compared to other alpha 2 adrenergic receptors that are not alpha 2a adrenergic receptors such as alpha 2b and alpha 2c receptors. The effects of the antagonist on an ADRA2a receptor may be specific as discussed above when compared to all other classes of adrenergic receptor that are present.

The specificity of the ADRA2a antagonist may apply within the whole body of the individual to be treated, that is the actions of the ADRA2a antagonist may be specific as discussed above throughout the body of the individual. The specificity of the ADRA2a antagonist may apply within particular tissues of the individual, such as the liver, kidneys or heart. That is, in one embodiment, the ADRA2a antagonist may act specifically to antagonise ADRA2a as discussed above within the liver of the individual being treated.

The ADRA2a antagonist may therefore be a specific antagonist of ADRA2a as described above. For example, the ADRA2a antagonist may not be an antagonist of ADRA2b, or may have no significant effect on the activity (e.g. signalling) or expression of ADRA2b. The ADRA2a antagonist may not be an antagonist of ADRA2c or may not have any significant effect on the activity or expression of ADRA2c. The ADRA2a antagonist may not be an antagonist of ADRA1 (alpha 1 adrenergic receptor) or may not have any significant effect on the activity or expression of ADRA1. The ADRA2a antagonist may not be an antagonist of beta adrenergic receptors (ADRB) or may not have any significant effect on the expression or activity of ADRB.

Any agent capable of inhibiting the activity or function of ADRA2a may be suitable for use in the methods of the present invention. Antagonists for use in accordance with the present invention may be direct or indirect antagonists of ADRA2a.

Direct antagonists are agents whose activity is directly on ADRA2a. For example, direct antagonists may be agents that act directly on the ADRA2a receptor to decrease its activity. A direct antagonist may be an agent that disrupts ADRA2a function or that destabilises the ADRA2a receptor. A direct antagonist may decrease the amount of ADRA2a by destroying or disrupting ADRA2a molecules within the patient. A direct antagonist may be an agent that acts on the ADRA2a gene, promoter or other gene regulatory regions to decrease expression of the ADRA2a. A direct antagonist may decrease expression of ADRA2a by preventing or reducing expression from the endogenous ADRA2a gene.

Any agent or molecule having the properties described above may be used as an ADRA2a antagonist in accordance with the present invention. Examples of ADRA2a antagonists or inhibitors that may be used in accordance with the present invention include:

BRL-44408 (2-[(4,5-Dihydro-1H-imidazol-2-yl)methyl]-2,3-dihydro-1-methyl-1H-isoindole), available from Sigma UK;

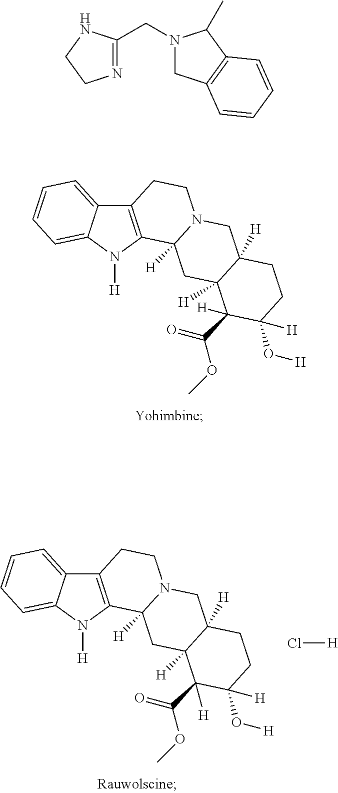 [SMILES: CC1c2ccccc2CN1CC1=NCCN1 ; COC(=O)[C@H]1[C@H]2C[C@H]3c4[nH]c5ccccc5c4CCN3C[C@H]2CC[C@@H]1O ; COC(=O)[C@H]1[C@H]2C[C@H]3c4[nH]c5ccccc5c4CCN3C[C@H]2CC[C@@H]1O ; Cl]

The ADRA2a antagonist may be a molecule that is capable of binding to, and preventing or disrupting the activity of ADRA2a.

Accordingly, one group of ADRA2a antagonists for use in accordance with this invention are anti-ADRA2a antibodies. Such an antibody may be monoclonal or polyclonal or may be an antigen-binding fragment thereof. For example, an antigen-binding fragment may be or comprise a F(ab)₂, Fab or Fv fragment, i.e. a fragment of the “variable” region of the antibody, which comprises the antigen binding site. An antibody or fragment thereof may be a single chain antibody, a chimeric antibody, a CDR grafted antibody or a humanised antibody.

An antibody may be directed to the ADRA2a molecule, i.e. it may bind to epitopes present on ADRA2a and thus bind selectively and/or specifically to ADRA2a. An antibody may be directed to another molecule that is involved in the expression and/or activity of ADRA2a. For example, a polyclonal antibody may be produced which has a broad spectrum effect against one or more epitopes on ADRA2a and/or one or more other molecules that are involved in the expression and/or activity of ADRA2a.

Antibodies can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

An antibody, or other compound, “specifically binds” to a molecule when it binds with preferential or high affinity to the molecule for which it is specific but does substantially bind not bind or binds with only low affinity to other molecules. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

The ADRA2a antagonist may be an antisense oligonucleotide, such as an antisense oligonucleotide against the gene encoding an ADRA2a protein.

The term “antisense oligonucleotide” as used herein means a nucleotide sequence that is complementary to the mRNA for a desired gene. Such an antisense oligonucleotide may selectively hybridise with the desired gene. In the context of the present invention, the desired gene may be the gene encoding ADRA2a.

The ADRA2a antagonist may modulate expression of the ADRA2a gene. For example, the ADRA2a antagonist may be a short interfering nucleic acid (siRNA) molecule, double stranded RNA (dsRNA), micro RNA, deoxyribose nucleic acid interference (DNAi) or short hairpin RNA (shRNA) molecule.

The term “selectively hybridise” as used herein refers to the ability of a nucleic acid to bind detectably and specifically to a second nucleic acid. Oligonucleotides selectively hybridise to target nucleic acid strands under hybridisation and wash conditions that minimise appreciable amounts of detectable binding to non-specific nucleic acids. High stringency conditions can be used to achieve selective hybridisation conditions as known in the art. Typically, hybridisation and washing conditions are performed at high stringency according to conventional hybridisation procedures. Washing conditions are typically 1-3×SSC, 0.1-1% SDS, 50-70° C. with a change of wash solution after about 5-30 minutes.

The ADRA2a antagonist may be a nucleic acid molecule such as an antisense molecule or an aptamer. The nucleic acid molecule may bind a specific target molecule.

Aptamers can be engineered completely in vitro, are readily produced by chemical synthesis, possess desirable storage properties, and elicit little or no immunogenicity in therapeutic applications. These characteristics make them particularly useful in pharmaceutical and therapeutic utilities.

The terms “nucleic acid molecule” and “polynucleotide” are used interchangeably herein and refer to a polymeric form of nucleotides of any length, either deoxyribonucleotides or ribonucleotides, or analogs thereof. A nucleic acid may comprise conventional bases, sugar residues and inter-nucleotide linkages, but may also comprise modified bases, modified sugar residues or modified linkages. A nucleic acid molecule may be single stranded or double stranded.

In general, aptamers may comprise oligonucleotides that are at least 5, at least 10 or at least 15 nucleotides in length. Aptamers may comprise sequences that are up to 40, up to 60 or up to 100 or more nucleotides in length. For example, aptamers may be from 5 to 100 nucleotides, from 10 to 40 nucleotides, or from 15 to 40 nucleotides in length. Where possible, aptamers of shorter length are preferred as these will often lead to less interference by other molecules or materials.

Aptamers may be generated using routine methods such as the Systematic Evolution of Ligands by EXonential enrichment (SELEX) procedure. SELEX is a method for the in vitro evolution of nucleic acid molecules with highly specific binding to target molecules. It is described in, for example, U.S. Pat. No. 5,654,151, U.S. Pat. No. 5,503,978, U.S. Pat. No. 5,567,588 and WO 96/38579. The SELEX method involves the selection of nucleic acid aptamers and in particular single stranded nucleic acids capable of binding to a desired target, from a collection of oligonucleotides. A collection of single-stranded nucleic acids (e.g., DNA, RNA, or variants thereof) is contacted with a target, under conditions favourable for binding, those nucleic acids which are bound to targets in the mixture are separated from those which do not bind, the nucleic acid-target complexes are dissociated, those nucleic acids which had bound to the target are amplified to yield a collection or library which is enriched in nucleic acids having the desired binding activity, and then this series of steps is repeated as necessary to produce a library of nucleic acids (aptamers) having specific binding affinity for the relevant target.

Any of the antagonists described herein may therefore be used to antagonise ADRA2a, i.e. to decrease the amount of ADRA2a that is present, and/or the activity (e.g. signalling via) or the function of the ADRA2a. This antagonism may take place in any location or tissue where ADRA2a is present. The antagonism may take place in one or more organs selected from the brain, kidney, liver and heart. The antagonism may take place on cells expressing ADRA2a, such as inflammatory cells, platelets and/or neurons. Preferably these antagonising effects take place in the liver.

An antagonist of ADRA2a may be an agent that decreases the production of endogenous ADRA2a. For example, the agent may act within the cells of the subject to inhibit or prevent the expression of ADRA2a. Such an agent may be a transcription factor or enhancer that acts on the ADRA2a gene to inhibit or prevent gene expression.

### Screening Methods

The present invention also provides methods for the identification of agents suitable for use in the treatment of liver disease. For example, the invention provides methods for the identification of antagonists of ADRA2a which are suitable for use in treating liver disease, such as in lowering portal pressure. Antagonists identified by this method may be antagonists of ADRA2a having any of the characteristics or effects described above. Antagonists identified by the methods described herein may be suitable for use in the treatment of liver disease or in the treatment or prevention of any of the conditions or symptoms described herein.

Accordingly, the invention provides a method of identifying an agent for use in the treatment of liver disease, the method comprising determining whether a test agent is capable of decreasing the activity or expression of ADRA2a. For example, the method may involve determining whether a test agent is capable of decreasing the amount or activity of ADRA2a, wherein the ability to decrease the amount or activity of ADRA2a indicates that the compound may be suitable for use in treating liver disease as described herein.

The method may comprise assessing the amount or activity of ADRA2a in a particular cell or tissue type. This may be any cell of tissue that expresses ADRA2a. For example, the method may assess the amount or activity of ADRA2a in the liver or in tissue or cells derived from the liver; in the kidney or heart or cells derived from the kidney or heart; in inflammatory cells, platelets or neurons; or in any other cell or tissue that expresses ADRA2a.

A test agent for use in a screening method of the invention refers to any compound, molecule or agent that may potentially antagonise ADRA2a. The test agent may be, or may comprise, for example, a peptide, polypeptide, protein, polynucleotide, small molecule or other compound that may be designed through rational drug design starting from known antagonists of ADRA2a.

The test agent may be any agent having one or more characteristics of an antagonist of ADRA2a as described above.

The test agent to be screened could be derived or synthesised from chemical compositions or man-made compounds. Candidate agents may be obtained from a wide variety of sources including libraries of synthetic or natural compounds. Suitable test agents which can be tested in the above assays include compounds derived from combinatorial libraries, small molecule libraries and natural product libraries, such as display (e.g. phage display) libraries. Multiple test agents may be screened using a method of the invention in order to identify one or more agents having a suitable effect on ADRA2a, such as inhibition of ADRA2a activity or expression.

The screening methods of the invention may be carried out in vivo, ex vivo or in vitro. In particular, the step of contacting a test agent with ADRA2a or with a cell or tissue that comprises ADRA2a may be carried out in vivo, ex vivo or in vitro. The screening methods of the invention may be carried out in a cell-based or a cell-free system. For example, the screening method of the invention may comprise a step of contacting a cell or tissue comprising ADRA2a with a test agent and determining whether the presence of the test agent leads to a decrease in the amount or activity of ADRA2a in the cell or tissue.

For example, the ability of a test agent to decrease the activity or expression of ADRA2a may be tested in a host cell or tissue that expresses ADRA2a. For example, the amount or activity of ADRA2a may be assessed in vitro, in vivo or ex vivo in the liver or in tissue or cells derived from the liver, in tissue or cells from another organ that expresses ADRA2a, such as the kidney or heart, or in other cells that express ADRA2a such as inflammatory cells, platelets or neurons.

In such a cell-based assay, the ADRA2a and/or the test agent may be endogenous to the host cell or tissue, may be introduced into a host cell or tissue, may be introduced into the host cell or tissue by causing or allowing the expression of an expression construct or vector or may be introduced into the host cell or tissue by stimulating or activating expression from an endogenous gene in the cell.

In such a cell-based method, the amount of ADRA2a may be assessed in the presence or absence of a test agent in order to determine whether the agent is altering the amount of ADRA2a in the cell or tissue, such as through regulation of ADRA2a expression in the cell or tissue or through destabilisation of ADRA2a protein within the cell or tissue. The presence of a lower ADRA2a activity (e.g. a descreased amount of signalling via ADRA2a) or a decreased amount of ADRA2a within the cell or tissue in the presence of the test agent indicates that the test agent may be a suitable antagonist of ADRA2a for use in accordance with the present invention in the treatment of an individual having liver disease.

In one embodiment, such a cell based assay may be carried out in vitro or ex vivo on cells or tissue deriving from the patient to be treated. It may therefore be determined whether or not the test agent is capable of decreasing the activity or amount of ADRA2a in the cells or tissue of that subject. For example, such a method may be carried out on a sample of cells or tissue from the liver of the patient.

A method of the invention may use a cell-free assay. For example, the ADRA2a may be present in a cell-free environment. A suitable cell-free assay may be carried out in a cell extract. For example, the contacting steps of the methods of the invention may be carried out in extracts obtained from cells that may express, produce or otherwise contain ADRA2a and/or a test agent. A cell-free system comprising ADRA2a may be incubated with the other components of the methods of the invention such a test agent.

In such a cell-free method, the amount of ADRA2a may be assessed in the presence or absence of a test agent in order to determine whether the agent is altering the amount of ADRA2a in the cell or tissue, such as through destabilisation of ADRA2a protein. In either case, the presence of a lower ADRA2a activity or a decreased amount of ADRA2a in the presence of the test agent indicates that the test agent may be a suitable antagonist of ADRA2a for use in accordance with the present invention in the treatment of an individual having liver disease.

The contacting step(s) of the method of the invention may comprise incubation of the various components. Such incubations may be performed at any suitable temperature, typically between 4° C. and 40° C. Incubation periods may be selected for optimum activity, but may also be optimized to facilitate rapid high-throughput screening. Following the contact and optional incubation steps, the subject methods may further include a washing step to remove unbound components, where such a washing step is generally employed when required to remove label that would give rise to a background signal during detection, such as radioactive or fluorescently labelled non-specifically bound components.

Incubation in cell or cell-free assay systems may be performed in a microtiter plate (e.g. a 96-well plate or other microwell plate). Further, incubation may be performed in an automated fashion (e.g. for high-throughput screening).

A screening method of the invention may be carried out in vivo. For example, a screening method may be carried out in an animal model. The animal model may be model characterised by portal hypertension. The animal model may be a model of cirrhosis. In such an in vivo model, the effects of a test agent may be assessed in the liver, or in other organs, cells or tissues that express ADRA2a such as the kidney or heart or in inflammatory cells, platelets or neurons. Preferably, the animal is a non-human animal such as a rat. For example, a screening method may be carried out in a bile duct-ligated rat model as described in the Examples. As shown in the Examples, bile duct ligation in the rat leads to an increase in ADRA2a levels in the liver of the rat. Such a model may therefore be suitable for identifying agents capable of decreasing ADRA2a levels. Accordingly, the screening method of the present invention may comprise the step of administering a test agent to a bile duct ligated rat and determining whether the presence of the test agent leads to a decrease in the amount or activity of ADRA2a in the liver or other organs, cells or tissues of the rat as discussed above.

Such a model may be used to assess the in vivo effects of a test agent. For example, such a model may be used to assess whether the test agent is capable of decreasing the activity or amount of ADRA2a in vivo. In such a method, the amount of ADRA2a may be assessed and/or the activity of ADRA2a, such as signalling by ADRA2a, may be assessed.

An in vivo model may also be used to determine whether the test agent has any unwanted side effects. For example, a method of the invention may compare the effects of a test agent on ADRA2a with its effects on other receptors in order to determine whether the test agent is specific.

In an in vivo model as described herein, or an in vitro model such as a cell-based or cell-free assay model as described herein, the effects of a test agent on ADRA2a may be compared with the effects of the same agent on other adrenergic receptors. As discussed above, a preferred ADRA2a antagonist for use in a method of treatment as described herein may be an agent that antagonises ADRA2a, but that does not antagonise other adrenergic receptors. The screening methods of the invention may thus include an additional step of assessing whether the test agent has any effect on the activity or amount of one or more other adrenergic receptors such as one or more adrenergic receptors that are not ADRA2a. In such a method, a test agent may be identified as a suitable ADRA2a antagonist if it is found to decrease the activity or a mount of ADRA2a, but not to decrease, not to significantly decrease, not to significantly decrease, not to alter, or not to significantly alter, the activity or amount of one or more other adrenergic receptors in the same assay. A test agent may be identified as a suitable ADRA2a antagonist if it meets any of the requirements discussed above for a selective ADRA2a antagonist of the present invention. For example, a suitable ADRA2a antagonist may not decrease the activity of one or more other adrenergic receptors, or may decrease the activity of other adrenergic receptor(s) to a lesser extent, such as a lower percentage decrease, such as less than 20%, less than 10%, less than 5%, less than 2%, less than 1% or less than 0.5% when compared to its effect on ADRA2a. The one or more other adrenergic receptors may be selected from one or more of alpha 2b, alpha 2c, alpha 1 and beta adrenergic receptors.

Where the assay is carried out in vivo, for example in a bile duct ligated rat model as described herein, such a method may comprise comparing the amount or activity of ADRA2a in the liver or other organs of the test animal in the presence or absence of the test agent. An observation that the level or activity of ADRA2a is decreased in the liver or other organs of animals treated with the test agent suggests that the test agent may be a suitable antagonist of ADRA2a. A further finding that treatment with the same test agent does not significantly decrease or alter the levels or activity of one or more other adrenergic receptors, such as ADRA2b or ADRA2c, may further indicate that the test agent is a suitable specific antagonist of ADRA2a that may be used in the methods of treatment described herein.

In the screening methods described herein, the presence of a lower ADRA2a activity or a decreased amount of ADRA2a in the presence of the test agent indicates that the test agent may be a suitable antagonist of ADRA2a for use in accordance with the present invention to treat an individual having liver disease, such as to lower portal pressure.

A test agent that is an antagonist of ADRA2a may result in a decrease in ADRA2a activity (e.g. signalling) or levels of at least 5%, at least 10%, at least 25%, at least 50%, at least 60%, at least 75%, or at least 85% or more in the presence of the test agent compared to in the absence of the test agent. A test agent that is an antagonist of ADRA2a may result in a decrease in ADRA2a activity or levels such that the activity or level of ADRA2a is no longer detectable in the presence of the test agent. Such a decrease may be seen in the sample being tested or, for example where the method is carried out in an animal model, in particular tissue from the animal such as in the liver.

Levels or amounts of ADRA2a may be measured by assessing expression of the ADRA2a gene. Gene expression may be assessed by looking at mRNA production or levels or at protein production or levels. Expression products such as mRNA and proteins may be identified or quantified by methods known in the art. Such methods may utilise hybridisation to specifically identify the mRNA of interest. For example such methods may involve PCR or real-time PCR approaches. Methods to identify or quantify a protein of interest may involve the use of antibodies that bind that protein. For example, such methods may involve western blotting. Regulation of ADRA2a gene expression may be compared in the presence and absence of a test agent. Thus test agents can be identified that decrease ADRA2a gene expression compared to the level seen in the absence of the test agent. Such test agents may be suitable antagonists of ADRA2a in accordance with the invention.

The effects of a test agent may be assessed by assessing the effects of that agent on a cell that expresses ADRA2a. The specificity of the agent may be assessed in a similar way, by assessing morphometry of the receptor on several cell types which express either only ADRA 2a, or other receptors that are not ADRA2a, such as ADRA2b or ADRA2c, and testing for downstream signals to determine specificity. Such experiments may be carried out using cell types that are known to express the various adrenergic receptor types. Such experiments may be carried out using cells that have been engineered to contain or express one or more adrenergic receptor types, such as ADRA2a that would not naturally be expressed by such cells.

### Pharmaceutical Formulations

A suitable ADRA2a antagonist as described herein is typically formulated for administration with a pharmaceutically acceptable carrier or diluent. The antagonist may be any antagonist as defined herein including any antagonist identified by a screening method of the invention. The antagonist may thus be formulated as a medicament with a standard pharmaceutically acceptable carrier(s) and/or excipient(s) as is routine in the pharmaceutical art. The exact nature of the formulation will depend upon several factors including the desired route of administration. Typically, the antagonist may be formulated for oral, intravenous, intragastric, intravascular or intraperitoneal administration.

The pharmaceutical carrier or diluent may be, for example, an isotonic solution such as physiological saline. Solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with ornithine and at least one of phenylacetate and phenylbutyrate, a pharmaceutically acceptable carrier, e.g. sterile water, olive oil, ethyl oleate, glycols, e.g. propylene glycol, and if desired, a suitable amount of lidocaine hydrochloride.

Where the antagonist to be administered is a nucleic acid molecule, for example where the antagonist is in the form of an expression vector, certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules.

A pharmaceutical formulation in accordance with the present invention may further comprise one or more additional therapeutic agents. For example, the formulation may comprise one or more ADRA2a antagonists as defined herein. The formulation may comprise one or more ADRA2a antagonists as described here and also one or more additional therapeutic agents. Preferably the additional therapeutic agent(s) are agents which will assist in the treatment or prophylaxis of the individual to be treated. For example, one or more agents that are effective at treating liver disease may be administered as part of a formulation as described herein. One or more agents that are effective at treating an underlying liver condition or symptom thereof in the patient may be administered as part of a formulation as described herein.

### Treatment

The present invention provides methods for the treatment of individuals having liver disease, particularly for the treatment of symptoms and conditions associated with or resulting from liver cirrhosis. Accordingly, the invention provides a method of treating an individual having liver disease comprising administering to said subject an antagonist of ADRA2a. Similarly, an antagonist of ADRA2a may be provided for use in a method of treating an individual having liver disease. Also provided is the use of an antagonist of ADRA2a in the manufacture of a medicament for use in the treatment of an individual having liver disease.

The antagonist may be any antagonist as described herein including any antagonist identified by a screening method of the invention. The antagonist may be provided in a formulation as described herein. An antagonist of ADRA2a as described herein is thus administered to a subject in order to treat liver disease, or particular symptoms or conditions associated with liver disease in the subject. An antagonist of ADRA2a as described herein can thus be administered to improve the condition of a subject, for example a subject suffering from liver disease or cirrhosis. An antagonist of ADRA2a as described herein may be administered to alleviate the symptoms of a subject, for example the symptoms associated with liver disease or cirrhosis. An antagonist of ADRA2a as described herein may be administered to combat or delay the onset of liver failure or of portal hypertension or any symptom associated therewith, such as varices. The invention can therefore prevent the medical consequences of cirrhosis. The individual may be at risk of liver failure, for example due to chronic liver disease such as cirrhosis. The methods described herein may be used to prevent or delay the onset of liver failure in such a patient, such as a patient having cirrhosis. Use of an antagonist of ADRA2a as described herein may thus extend the life of a patient with liver disease.

The treatment of liver disease, or the treatment of an individual having liver disease, as described herein, refers to the treatment of an individual having liver disease. The individual may be suffering from liver failure, such as acute liver failure (ALF) or acute on chronic liver failure (ACLF). The individual may be suffering from chronic liver disease such as cirrhosis. The individual may be suffering from alcoholic liver disease (e.g. which may include hepatitis) or non-alcoholic fatty liver disease. The methods described herein may be used in the prevention or treatment of any such disease.

The individual may be suffering from, or at risk of, one or more symptoms or conditions caused by or associated with liver disease or cirrhosis. Any one or more of these conditions or symptoms may be treated or prevented in accordance with the present invention. For example, the individual may be suffering from, or at risk of, one or more of the following as a result of their liver disease or cirrhosis: peripheral vasodilation (e.g. associated with normal, reduced or increased cardiac output); splanchnic vasodilation (e.g associated with normal, reduced or increased cardiac output); reduced cardiac output, portal hypertension, reduced mean arterial pressure, reduced hepatic arterial blood flow, increased intrahepatic resistance, increased plasma ammonia, increased brain water, increased plasma creatinine, renal dysfunction, hepato-renal dysfunction, increased plasma lactate, increased plasma alanine and/or aspartate aminotransferase, alcoholic liver disease, non-alcoholic fatty liver disease and/. The methods and uses described herein may be of utility in the treatment or prevention of any one or more of these symptoms or conditions in an individual suffering from liver disease.

As described herein, the antagonist of ADRA2a may lead to decreased expression and/or decreased levels of ADRA2a in the liver of the subject. For example, the antagonist may be an agent that inhibits transcription of ADRA2a in cells of the subject.

As described herein, the antagonist of ADRA2a may lead to decreased activity of ADRA2a in the liver of the individual.

The subject is treated with an antagonist of ADRA2a as described herein. As described above, the antagonist of ADRA2a may be administered alone or in the form of a pharmaceutical formulation. The formulation may comprise one or more antagonists of ADRA2a and may comprise one or more additional therapeutic or prophylactic agents.

Two or more different ADRA2a antagonists as described herein may be used in combination to treat a subject. The two or more antagonists may be administered together, in a single formulation, at the same time, in two or more separate formulations, or separately or sequentially as part of a combined administration regimen.

An antagonist or formulation of the invention may be administered by any suitable route. Preferably it is administered by oral, intravenous, intragastric, intraperitoneal or intravascular routes. The antagonist or formulation may be administered directly to the liver of the subject.

The antagonist is administered in a therapeutically effective amount. A suitable dose of an antagonist of the invention can be determined according to various parameters such as the age, weight and condition of the subject to be treated; the type and severity of the liver disease; the route of administration; and the required regimen. A suitable dose can be determined for an individual antagonist. For example, for some antagonists a typical dose may be in the order of from 1 mg/kg/day to 30 g/kg/day. A physician will be able to determine the required dosage of antagonist and for any particular subject.

The present invention is broadly applicable to therapeutic methods and is relevant to the development of prophylactic and/or therapeutic treatments. It is to be appreciated that all references herein to treatment include curative, palliative and prophylactic treatment.

Prophylaxis or therapy includes but is not limited to eliciting an effective decrease in ADRA2a amount, function or activity in order to cause a reduction in one or more symptoms or conditions associated with, or resulting from, liver disease or cirrhosis. The symptoms or conditions may be, for example, any of those discussed above. For example, prophylaxis or therapy may result in: increased cardiac output; reduced portal pressure; increased mean arterial pressure; increased hepatic arterial blood flow; decreased intrahepatic resistance; decreased plasma ammonia; improved renal function, decreased brain water; decreased plasma creatinine, decreased plasma lactate. Prophylaxis or therapy may result in the prevention or delay of such symptoms, such as the prevention or delay of liver failure in an individual at risk of such liver failure, such as an individual with cirrhosis. Prophylaxis or therapy may result in the maintenance of a particular level of cardiac output mean arterial pressure and/or hepatic arterial blood flow in a patient where symptoms have been increasing or are expected to increase as a result of the liver disease and/or cirrhosis. Prophylaxis or therapy may result in the maintenance of a particular level of portal pressure, intrahepatic resistance, plasma ammonia, renal function, brain water, creatinine, plasma lactate and plasma aminotransferases in a patient where such factors have been increasing or in which such factors are expected to increase as a result of the liver disease and/or cirrhosis. Prophylaxis or therapy may result in such changes in symptoms or conditions in such an individual changing at a reduced rate compared to the changes that would have been seen or would have been expected in the absence of such treatment.

Prophylaxis or therapy may have similar effects in relation to any of the symptoms or consequences of liver disease or cirrhosis described herein. That is, treatment in accordance with the present invention may lead to a lessening in the severity of such symptoms or consequences, maintenance of an existing level of such symptoms or consequences or a slowing or reduction in the worsening of such symptoms or consequences.

### Patients to be Treated

The present invention relates to the treatment of liver disease such as cirrhosis in individuals in need thereof. An individual to be treated in accordance with the present invention may therefore have liver disease such as cirrhosis or may be at increased risk of liver disease such as cirrhosis. For example, the subject may have cirrhosis. The subject may have portal hypertension. Portal hypertension may be defined as increased blood pressure in the portal vein and its tributaries. The portal vein is the large vein that brings blood from the intestine to the liver. Portal hypertension may be defined as clinically significant when the portal pressure gradient (the difference in pressure between the hepatic veins or the portal vein (e.g. measurements of a catheter wedged in the portal vein or hepatic veins) and the hepatic vein or inferior vena cava (e.g. the free pressure readings in the hepatic vein or inferior vena cava) of 5 mm Hg of reater, preferably 10 mm Hg or greater.

Methods for diagnosing portal hypertension are well known in the art and in particular to clinicians and veterinarians in the field. Preferably, the subject will have been diagnosed as having portal hypertension, for example by a medical or veterinarian professional. The subject may display one or more symptoms associated with portal hypertension. Portal pressure may be measured directly. For example, a catheter may be inserted through an incision in the neck and threaded through blood vessels into the liver or spleen to directly measure pressure in the portal blood vessels.

The individual to be treated may have been diagnosed as suffering from cirrhosis, or one or more symptoms or conditions as described herein that may be associated with cirrhosis, for example by any of these methods. The individual to be treated may have been diagnosed as being at risk of cirrhosis or such symptoms or conditions. For example, the individual may have been diagnosed with one or more symptoms that are associated with cirrhosis. For example, the individual to be treated may have liver cirrhosis, alcoholic hepatitis, idiopathic non-cirrhotic portal hypertension, congenital hepatic fibrosis, partial nodular transformation, Budd-Chiari syndrome, portal vein thrombosis, right heart failure or schistosomiasis infection. The methods described herein may be used to prevent liver failure in a patient having cirrhosis.

The individual to be treated may be identified as having one or more of the following indications: acute on chronic liver failure, acute liver failure, alcoholic liver disease, and non-alcoholic fatty liver disease, non-alcoholic steatohepatitis. An individual suffering from any of these conditions may be treated in accordance with the present invention. The present invention may be used to treat, prevent or ameliorate the effects of any of these conditions.

The individual to be treated may not be suffering from inflammation or from inflammatory symptoms. For example, the individual may not be suffering from active hepatitis or from other liver inflammation. The individual may have no significant or no detectable inflammation of the liver or no inflammation of the liver associated with the condition to be treated. The individual may fail to show elevated levels of one or more inflammatory markers such as C-reactive protein, serum amyloid A, TNFα, IL-6, IL-8 or IL-18.

The subject to be treated may be any individual which is susceptible to increased portal pressure such as portal hypertension. The subject may be male or female. Women may be more susceptible to the adverse effects of alcohol than men. Women can develop alcoholic chronic liver disease in a shorter time frame and from smaller amounts of alcohol than men.

The subject to be treated may be a human. The subject to be treated may be a non-human animal. The subject to be treated may be a farm animal for example, a cow or bull, sheep, pig, ox, goat or horse or may be a domestic animal such as a dog or cat. The subject may or may not be an animal model for liver disease. The animal may be any age, but will often be a mature adult subject.

### EXAMPLES

### Example 1

### Noradrenaline Levels in Liver Failure Patients

Patients were classified as compensated, decompensated or acute on chronic liver failure (ACLF) using standard criteria. Patients were assessed for levels of noradrenaline, portal pressure (HVPG) and hepatic blood flow.

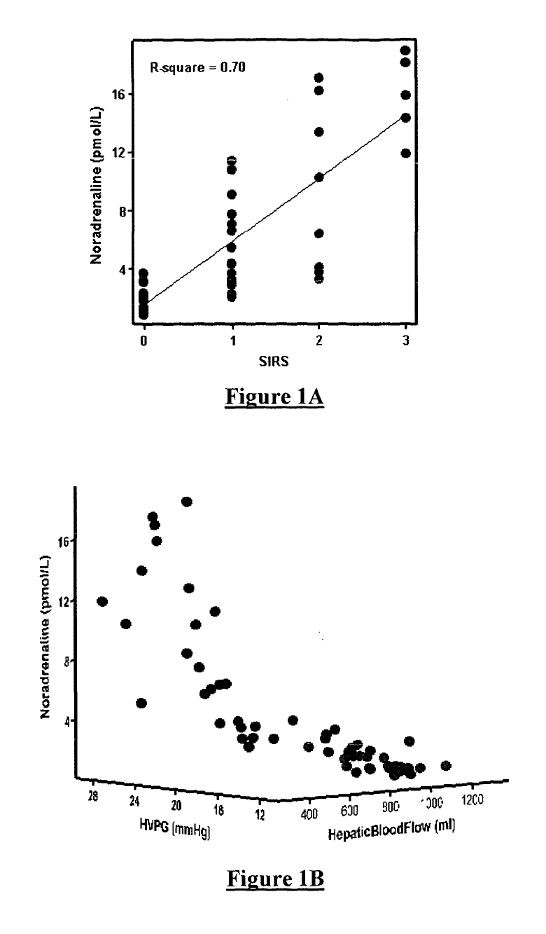

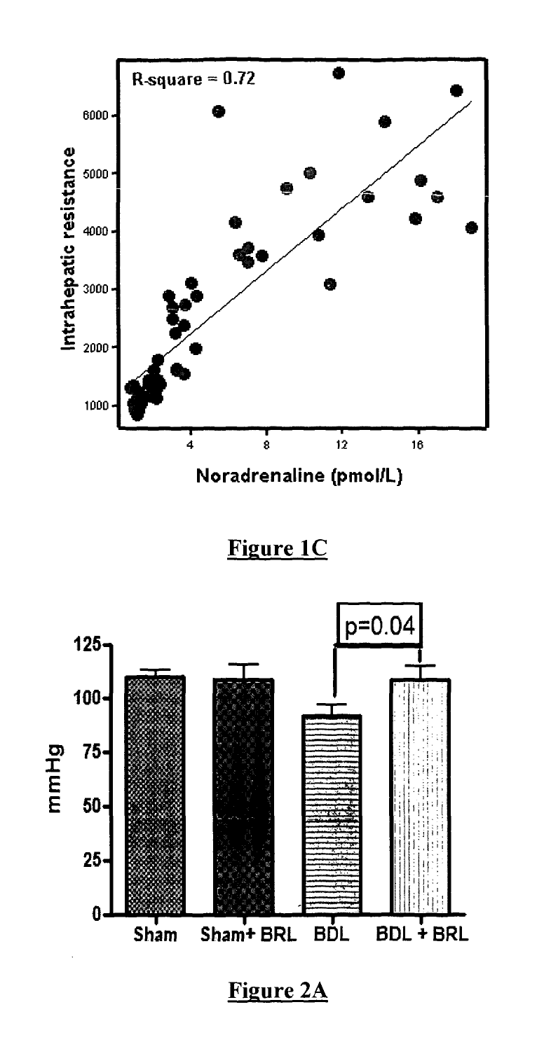

As shown in FIG. 1, higher noradrenaline levels correlated with higher portal pressure (HVPG) and with lower hepatic blood flow. This indicates the existence of higher intra hepatic resistance as the disease progress.

### Example 2

### Effects of Alpha 2a Antagonist

### Methods

These experiments utilised an established animal model of cirrhosis, the bile duct ligated (BDL) rat. BDL rats may be generated by methods known in the art. For example, male Sprague-Dawley rats (200-250 g) may be used for this procedure. Following anaesthetisation, a mid-line laparotamy may be performed, the bile duct exposed, triply ligated with 4.0 silk suture, and severed between the second and third ligature. The wound is then closed in layers with absorbable suture, and the animal allowed to recover in a quiet room before being returned to the animal storage facility.

29 bile duct ligated rats and 17 sham operated rats were studied at the end of four weeks. The animals were randomised to receive a placebo (saline) or an alpha 2a antagonist (BRL 44408, Sigma, UK). The dose applied was 10 mgs/Kg by sub cutaneous injection: two doses were delivered 24 hours prior to study.

Mean arterial pressure was measured via a transducer through the left carotid artery. Portal pressure readings were taken by direct puncture of portal vein and transducer. Portal vein and hepatic artery flow measurements were done with a transonic flow probe and meter. All measurements were done in triplicates and recorded in data collection sheets.

Plasma biochemistry was measured by colorimetry using a Cobas Integra. Alpha 2a adrenergic receptor (ADRA 2a) and Nuclear Factor κ B (NFκB) protein expression were assessed by western blotting. ADRA2a expression was determined by immunohistochemistry.

Data are shown as median (range) or mean±standard error (SEM). Two tailed unpaired t-test was used to define difference between the means of normally distributed data of equal variance. For data that was not equally distributed, a Mann-Whitney test was used. Comparison of multiple groups was by analysis of variance (ANOVA) with a Bonferroni correction for multiple comparison.

### Results

Following treatment with ADRA2a antagonist there was a significant increase in the MAP (p<0.05) and a significant reduction in portal pressure as compared to the placebo treated group (11.4±3.4 vs. 18.0±3.7 mmHg, p<0.001).

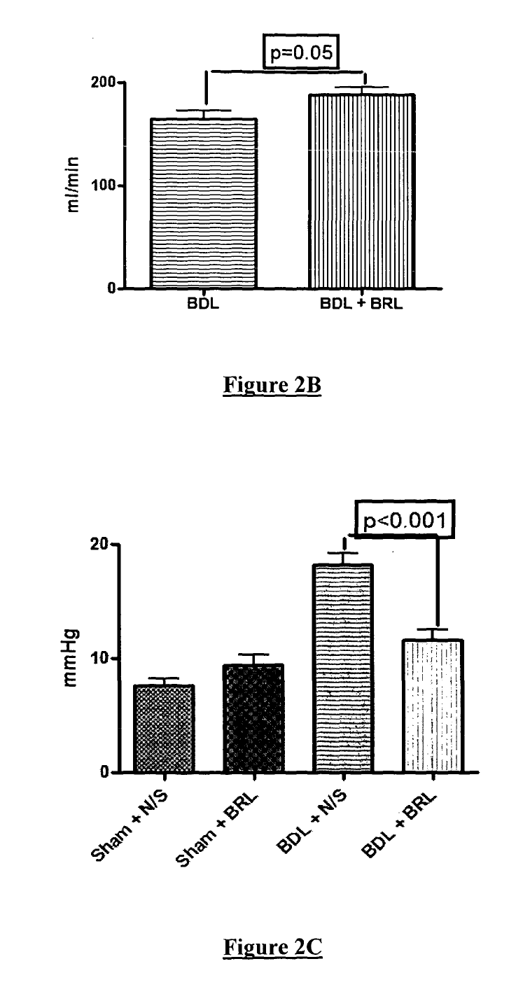

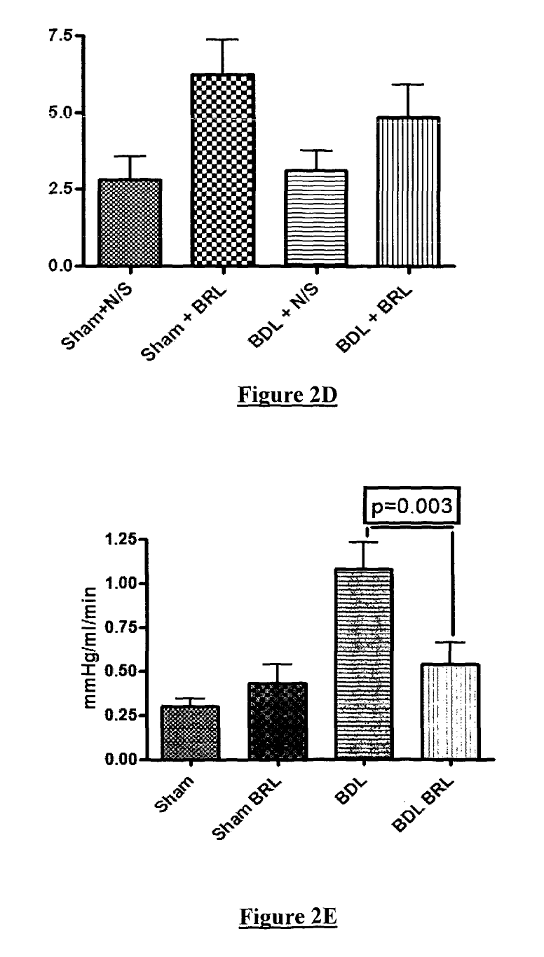

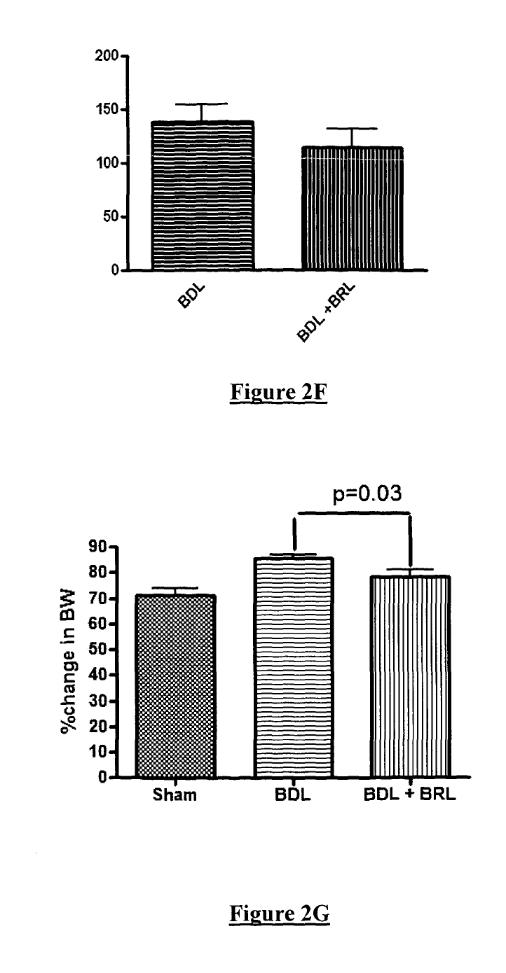

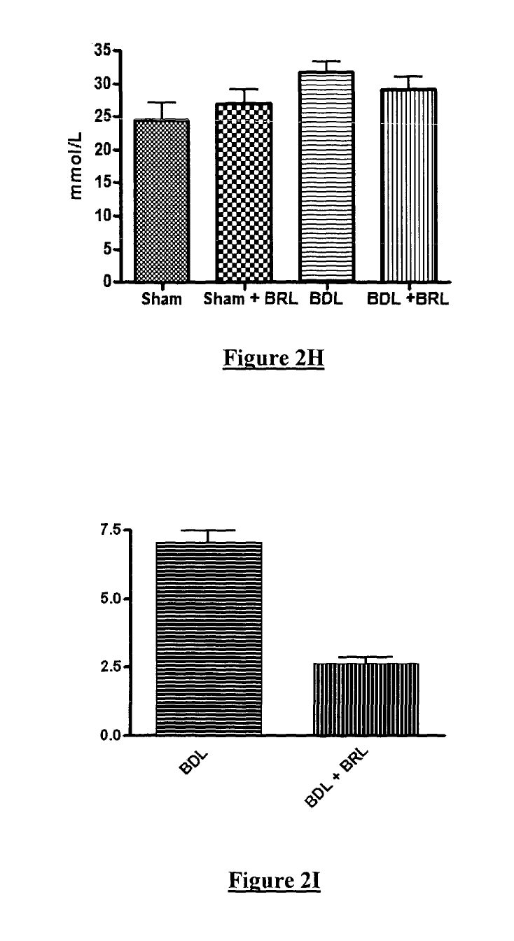

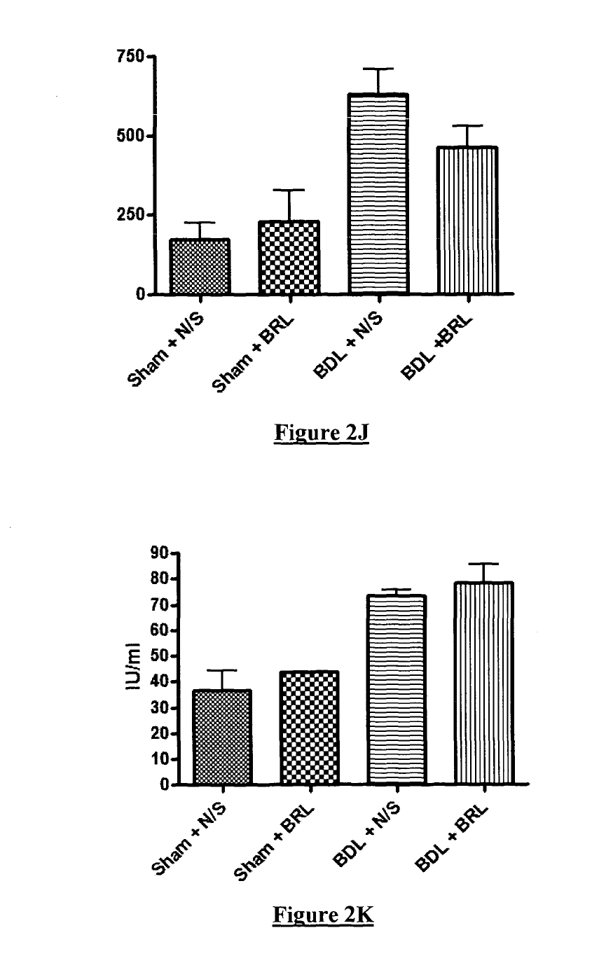

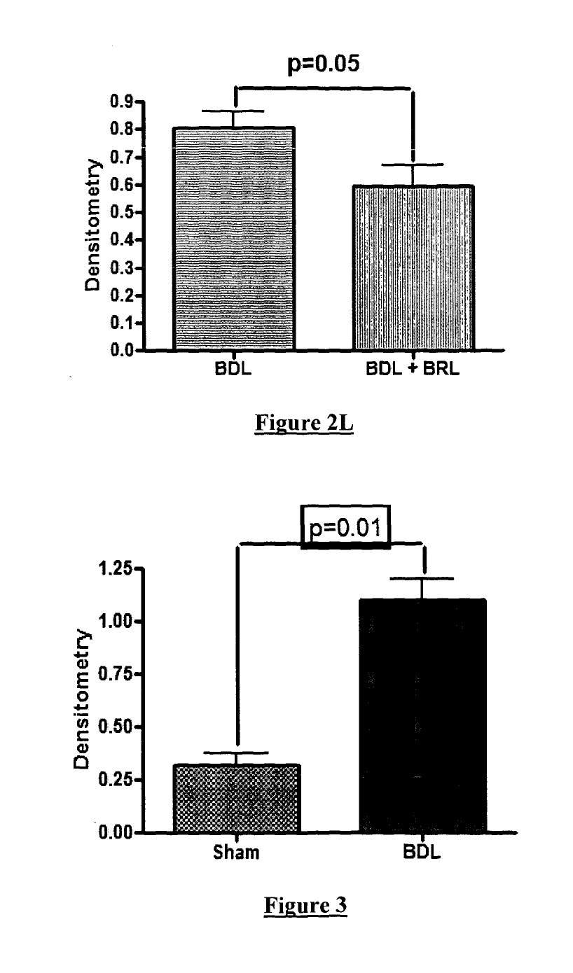

As shown in FIG. 2A, mean arterial pressure (MAP) was significantly improved in bile duct ligated animals treated with BRL 44408 (alpha 2a antagonist) [N=7] as compared to the untreated bile duct ligated (BDL) [N=9] rats. There was no difference in the MAP between the sham operated rats [N=4] and the sham operated rats treated with BRL 44408. The MAP was measured by a transducer through the left carotid artery and measured in triplicates after a period of stabilisation of 3 minutes.

As shown in FIG. 2B, cardiac output (a measure of the function of the heart) was improved significantly in BDL rats treated with BRL 44408 [N=7] as compared to untreated BDL rats [N=7]. The cardiac output was calculated by multiplying the heart beat rate per minute by the stroke volume (volume of blood ejected out by each beat). These values were obtained by a trans-throracic echocardiogram.

As shown in FIG. 2C, portal pressure was significantly reduced in the BDL rats treated with BRL 44408 [N=14] as compared to BDL rats treated with Saline [N=15]. There was no difference in the portal pressure between sham rats treated with BRL 44408 [N=7] and BDL treated with saline [N=8]. The portal pressure was measured by direct puncture of the portal vein and through a pressure transducer. Readings were done in triplicate after a period of stabilisation of three minutes.

The hepatic arterial blood flow was markedly increased in the treated group without significant change in the portal venous blood flow resulting in a significant reduction in intrahepatic resistance post treatment (1.1±0.2 vs. 0.5±0.1 mmHg/ml/min, p<0.05).

As shown in FIG. 2D, hepatic arterial blood flow was improved in the BDL rats treated with BRL44408 [N=7] as compared to BDL treated with saline [N=7]. There was improvement in the hepatic arterial blood flow in the sham operated rats treated with BRL44408 [N=3] as compared to sham rats treated with saline [N=4]. The hepatic arterial blood flow was measured with a Transonic flow probe on the hepatic artery after a period of stabilisation of 10 minutes and readings were taken in triplicate.

As shown in FIG. 2E, the intra hepatic resistance was significantly reduced in the BDL rats treated with BRL 44408 [N=6] as compared to BDL rats [N=6]. There was no difference in the intra hepatic resistance between the sham rats treated with BRL 44408 [N=3] as compared to sham rats [N=4]. The intra hepatic resistance was calculated by dividing the portal pressure by the hepatic blood flow.

As shown in FIG. 2F, plasma ammonia showed a trend towards reduction in the BDL rats treated with BRL 44408 [N=10] as compared to BDL rats [N=15]. Higher plasma ammonia has been shown to have adverse outcome in cirrhosis as it contributes to hepatic encephalopathy which is one of the major complication of cirrhosis.

As shown in FIG. 2G, the brain water content was significantly decreased in the BDL rats treated with BRL 44408 [N=7] as compared to the BDL rats [N=6]. The BDL rats had a significantly increased brain water content as compare with the sham rats [N=4]. The brain water content signifies the amount of brain swelling that occurs as a result of inflammation in the brain. It has been shown to be indicative of a worsened prognosis.

Biochemical analysis showed a significant reduction in plasma lactate (p<0.05), AST (p<0.05) and a trend towards reduction in creatinine in treated animals.

As shown in FIG. 2H, the plasma creatinine level was improved in the BDL rats treated with BRL 44408 [N=13] as compared to BDL rats [N=16]. There was no difference between the sham rats treated with BRL 44408 [N=6] as compared to sham operated rats [N=7]. Plasma creatinine is a marker for renal function.

As shown in FIG. 2I, the plasma lactate level was significantly reduced in the BDL rats treated with BRL [N=4] as compared to BDL rats [N=4]. Lactate is an indirect measurement of organ perfusion as it goes up in the context of loss of blood flow to any part of the body and/or in the context of metabolic stress.

As shown in FIG. 2J, plasma aspartate aminotransaminase (AST) levels were reduced (p=0.13) in the BRL 44408 treated BDL rats [N=9] as compared to the BDL rats [N+12]

As shown in FIG. 2K, there was also a non significant increase (p=0.5) in liver specific alanine aminotransferase (ALT).

As shown in FIG. 2L, NFkB protein expression in the liver tissue was reduced in the BDL rats treated with BRL 44408 [N=5] as compared to the BDL rats [N=5]. The protein expression was assessed by the western blotting technique. NFkB is an important regulator of cell signalling pathways including apoptosis and inflammation.

BDL rats had significantly increased hepatic protein expression of ADRA2a compared with sham operated rats and this was mostly shown to be located on hepatocytes by immunohistochemistry.

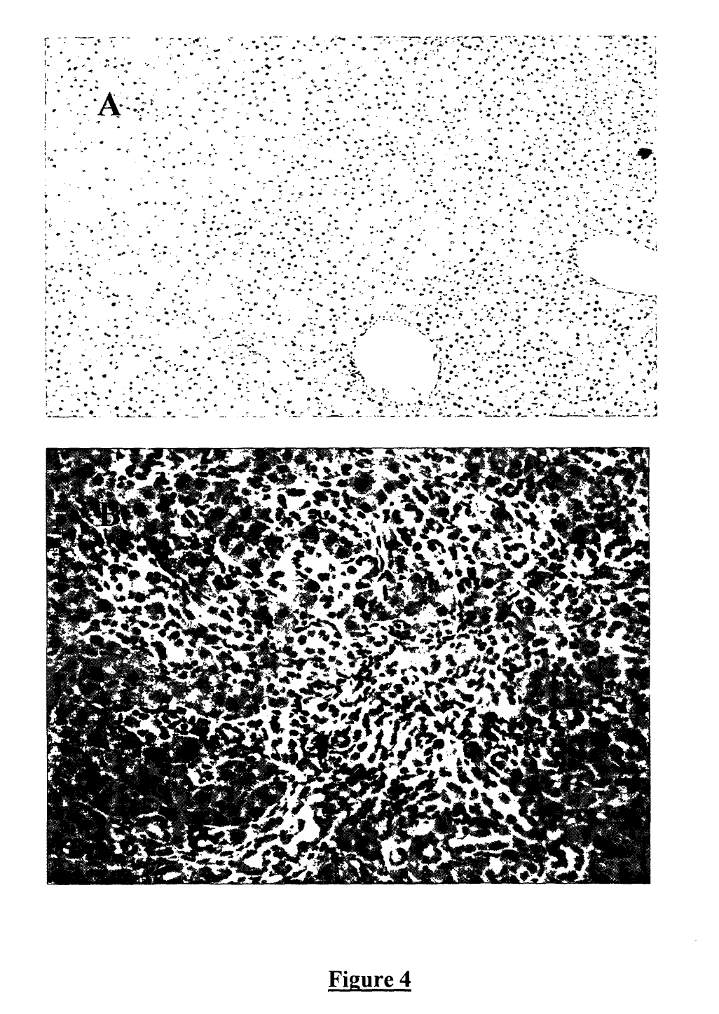

As shown in FIG. 4A, there was absence of expression of alpha 2a receptor in the sham operated rat as measured by immunohistochemistry. This indicates there is no alpha2a receptor expression in the normal rat liver. However, as shown in FIG. 4B, there was significant expression of alpha 2a receptor in the bile duct ligated rat liver. This indicates that the alpha 2a receptor may be over expressed in the context of liver disease/cirrhosis in bile duct ligated rat liver.

### CONCLUSION

The normal liver was found to have very limited expression of α2a adrenergic receptor, whereas the liver of BDL rats (a model for cirrhosis) showed a marked increase in expression.

Treatment of BDL rats with the alpha 2a adrenergic receptor antagonist BRL 44408 had a number of effects, such as:

lowered portal pressure;

increased MAP and hepatic artery blood flow;

lowered Intra-hepatic resistance; and

reduced ammonia, brain swelling, and renal dysfunction.

These effects are all useful in the treatment of liver disease, in particular the treatment of the consequences of cirrhosis such as the treatment of portal hypertension.

1. A method of treating liver disease in an individual in need thereof, said method comprising a step of administering an antagonist of an alpha 2a adrenergic receptor (ADRA2a).

2. The method according to claim 1, wherein said individual is suffering from chronic liver disease.

3. The method according to claim 1, wherein said individual is suffering from liver cirrhosis.

4. The method according to claim 1, wherein said method is for treating or preventing liver failure.

5. The method according to claim 1, wherein the individual is suffering from, or is at risk of one or more of the following, when compared to a subject not suffering from liver disease: (a) splanchnic vasodilation and normal, reduced or increased cardiac output; (b) portal hypertension; (c) reduced mean arterial pressure; (d) reduced hepatic arterial blood flow; (e) increased intra-hepatic resistance; (f) increased plasma ammonia; (g) hepato-renal dysfunction; (h) increased brain water; (i) increased plasma creatinine; (j) increased plasma lactate; (k) alcoholic cirrhosis; and/or (l) non-alcoholic fatty liver disease.

6. The method according to claim 1, wherein either or both of: (i) the individual is not suffering from active hepatitis, and (ii) the individual has no significant inflammation of the liver.

7. The method according to claim 1, wherein said antagonist leads to one or more of:
(a) decreased expression of ADRA2a in the liver of the individual;
(b) decreased levels of ADRA2a in the liver of the individual;
(c) decreased activity of ADRA2a in the liver of the individual;
(d) decreased signalling via ADRA2a in the liver of the individual.

8. The method according to claim 1, wherein said antagonist is selected from BRL-44408, Yohimbine and Rauwolscine.

9. The method according to claim 1, wherein said antagonist is one or more of:
(a) a specific antagonist of ADRA2a;
(b) a selective antagonist of ADRA2a;
(c) not an antagonist of ADRA2b;
(d) not an antagonist of ADRA2c;
(e) not an antagonist of ADRA1;
(f) not an antagonist of ADRB.

10. (canceled)

11. A method of identifying an agent for treating liver disease, the method comprising determining whether a test agent is capable of decreasing an ADRA2a amount or an ADRA2a activity, wherein an ability to decrease the amount or activity of ADRA2a indicates that the agent is suitable for treating liver disease.

12. The method according to claim 11, wherein the amount or activity of ADRA2a is assessed in one or more of:
(a) liver, or in tissue or cells derived from the liver;
(b) kidney or heart or cells derived from the kidney or heart;
(c) platelets or neurons; and
(d) another cell or tissue that expresses ADRA2a.

13. The method according to claim 11, comprising the step of administering a test agent to an animal model of portal hypertension or cirrhosis and determining whether presence of the test agent leads to a decrease in the amount or activity of ADRA2a in liver of a rat.

14. The method according to claim 13, wherein the animal model is a bile duct ligated rat.

15. The method according to claim 12, comprising the step of administering a test agent to an animal model of portal hypertension or cirrhosis and determining whether presence of the test agent leads to a decrease in the amount or activity of ADRA2a in liver of a rat.

16. The method according to claim 15, wherein the animal model is a bile duct ligated rat.
